(19) United States
(12) Patent Application Publication
(10) Pub. No.: US 2024/0404654 A1
(43) Pub. Date: Dec. 5, 2024

(54) DYNAMIC MONITORING METHOD FOR REBOUND RATE OF WET SPRAYING SHOTCRETE

(71) Applicants: CHINA TIESIJU CIVIL ENGINEERING GROUP CO., LTD., Anhui (CN); THE SEVENTH ENGINEERING CO.LTD OF CHINA TIESIJU CIVIL ENGINEERING GROUP, Shandong (CN); Wuhan Institute of Geotechnical Mechanics, Chinese Academy of Sciences, Wuhan (CN)

(72) Inventors: Haoming Wang, Anhui (CN); Chengyuan Zhang, Wuhan (CN); Yanwei Yang, Anhui (CN); Yong Wang, Anhui (CN); Jun Wang, Anhui (CN); Jianbin Yan, Anhui (CN); Haibo Wang, Anhui (CN); Xinghuo XU, Anhui (CN); Feng Ling, Anhui (CN); Zhipeng Li, Anhui (CN); Xianming Lin, Anhui (CN); Wei Liu, Anhui (CN); Yuanyuan Yang, Anhui (CN)

(73) Assignees: CHINA TIESIJU CIVIL ENGINEERING GROUP CO., LTD., Anhui (CN); THE SEVENTH ENGINEERING CO.LTD OF CHINA TIESIJU CIVIL ENGINEERING GROUP, Shandong (CN); Wuhan Institute of Geotechnical Mechanics, Chinese Academy of Sciences, Wuhan (CN)

(21) Appl. No.: 18/430,545

(22) Filed: Feb. 1, 2024

(30) Foreign Application Priority Data
May 29, 2023 (CN) 202310623124.4

(51) Int. Cl. G16C 60/00; E21D 11/10; G06F 30/28; G06F 111/10; G06F 113/08; G06F 113/26

(52) U.S. Cl. CPC G16C 60/00; E21D 11/105; G06F 30/28; G06F 2111/10; G06F 2113/08; G06F 2113/26

(57) ABSTRACT

A dynamic monitoring method for a rebound rate of wet spraying shotcrete includes converting a set of laser points obtained from scanning to a rock wall scanning surface relative coordinate system to form an initial geometric model of the rock face, calculating the initial rock wall volume, coarse spraying and fine spraying the shotcrete and performing the three-dimensional modeling calculations, and calculating the coarse spray rebound rate and the fine spray rebound rate, thereby solving the problem that at present, the estimated idealized data does not match the actual usage data, not only it is insufficient in quantification, but also the dynamic rebound rate of the wet spraying process is not understood, in which the rebound rate is inaccurately estimated, making it difficult to guide the adjustment of the shotcrete formula and the adjustment of the spray posture and speed, and thus the goal of process optimization cannot be achieved.

### CROSS-REFERENCE TO RELATED APPLICATION

This application claims the priority benefit of China application serial no. 202310623124.4, filed on May 29, 2023. The entirety of the above-mentioned patent application is hereby incorporated by reference herein and made a part of this specification.

### BACKGROUND

### Technical Field

The disclosure relates to the technical field of engineering measurement, and in particular to a dynamic monitoring method for a rebound rate of a wet spraying shotcrete.

### Description of Related Art

Wet spraying shotcrete in tunnel projects is a commonly used construction method. For example, preliminary support after drill-and-blast construction requires a large amount of shotcrete to be sprayed on the exposed rock surface to form a protective layer to prevent further deterioration of the rock layer. If the amount of shotcrete that is sputtered off is too large, then not only the efficiency of the construction is reduced, but also the economic benefit is greatly affected. Therefore, it is necessary to monitor and evaluate the rebound rate of shotcrete under various spraying conditions.

At present, the monitoring of the rebound rate of the wet spraying shotcrete is mainly roughly evaluated based on the design volume and the final usage. Based on the known size data of the tunnel design, total shotcrete consumption may be estimated based on the thickness of the shotcrete that needs to be formed after excavation. However, when the estimated idealized data does not match the actual usage data, not only it is insufficient in quantification, but also the dynamic rebound rate of the entire wet spraying process is not understood, in which the rebound rate is inaccurately estimated, making it difficult to guide the adjustment of the shotcrete formula and the adjustment of the spray posture and the spray speed, and thus the goal of process optimization cannot be achieved.

### SUMMARY

The purpose of the disclosure is to provide a dynamic monitoring method for a rebound rate of a wet spraying shotcrete, which solves the problem that the current monitoring of the rebound rate of the wet spraying shotcrete is not only insufficient in quantification, but also the dynamic rebound rate of the entire wet spraying process is not understood, in which the rebound rate is inaccurately estimated, making it difficult to guide the adjustment of the shotcrete formula and the adjustment of the spray posture and spray speed, and thus the goal of process optimization cannot be achieved.

In order to achieve the above purpose, the disclosure provides the following technical solution, a dynamic monitoring method for a rebound rate of wet spraying shotcrete, and the specific steps are as follows.
S1. Target balls are arranged in a rectangular shape on a rock surface, and the rock surface before wet spraying the shotcrete is scanned.
S2. A set of laser points obtained from scanning is converted to a rock wall scanning surface relative coordinate system.
S3. Scanned data in a local coordinate system is fused to form an initial geometric model of the rock surface and an initial rock wall volume is calculated.
S4. First, the shotcrete is coarse sprayed, and a three-dimensional modeling calculation of a coarse spray rebound rate is performed. Then, the shotcrete is fine sprayed, and a three-dimensional modeling calculation of a fine spray rebound rate is performed.
S5. The coarse spray rebound rate and the fine spray rebound rate are calculated.

Preferably, in S1, the specific steps of arranging the target ball in a rectangular shape on the rock surface include the following.

Target balls T1, T2, T3, and T4 are arranged in a rectangular shape on a rock surface to be studied. T1 and T2 are of the same height, while T3 and T4 are vertically above T1 and T2 respectively.

A fixed laser scanner is arranged on each side of the wet spray machine and pointing to the same area to be scanned comprising the reflective target balls.

Preferably, in S1, the specific steps of scanning the rock surface before wet spraying the shotcrete include the following.

The rock surface before wet spraying the shotcrete is scanned by using the first scanner and the second scanner respectively.

Coordinates of the target ball T1 and the target ball T2 obtained by the first scanner are recorded as T1A (X_{AT1}, Y_{AT1}) and T2A (X_{AT2}, Y_{AT2}) respectively. The distance between T1 and T2 are calculated and recorded as L_{T1T2}.

The distance:

$${L}_{T 1 T 2} = \sqrt{{( {X}_{T 1 A} - {X}_{T 2 A} )}^{2} + {( {Y}_{T 1 A} - {Y}_{T 2 A} )}^{2}}$$

The rock wall scanning surface relative coordinate system is established by using T1 and T2. T1 is used as an origin, a direction of T1 pointing to T2 is a positive direction of an X-axis, a vertical direction on a horizontal plane is a positive direction of a Y-axis, a vertical direction upward is a positive direction of a Z-axis, coordinates of T1 are (0,0,0), coordinates of T2 are (L_{T1T2},0,0).

Coordinates of T3 are (0,0,H), coordinates of T4 are (L_{T1T2},0,H), and H is a numerical value where T3 and T4 are higher than T1 and T2.

Preferably, the specific steps of converting the set of laser points obtained from the first scanner in S2 to the rock wall scanning surface relative coordinate system include the following.

Point coordinates of a measuring point S on the rock wall and shotcrete surface measured by a scanner A are recorded as SA(x_{A}, y_{A}, z_{A})

Coordinates of T1 and T2 are recorded as T₁(x_{AT1}, y_{AT1}, z_{AT1}) and T₂(x_{AT2}, y_{AT2}, z_{AT2}).

Coordinates of a laser scanning point SA in the scanning surface relative coordinate system established in S2 is SA(x_{new}, y_{new}, z_{new}).

Then, the new coordinates of the X-axis are calculated as:

$${x}_{new} = \sqrt{( {( {x}_{1} - {x}_{A T 1} )}^{2} + {( {y}_{1} - {y}_{A T 1} )}^{2} )}$$

In which

$${x}_{1} = ( k \cdot {y}_{A} + {x}_{A} - k \cdot {x}_{A} - {y}_{A} / k ) / ( {k}^{2} + 1 )$$

$${y}_{1} = ( {k}^{2} \cdot {y}_{A} + k \cdot {x}_{A} + {y}_{A T 1} - k \cdot {x}_{A T 1} - k \cdot {x}_{A} - {y}_{A} ) / ( {k}^{2} + 1 )$$

$$k = ( {y}_{A T 2} - {y}_{A T 1} ) / ( {x}_{A T 2} - {x}_{A T 1} )$$

The new coordinates of the Y-axis are:

$${y}_{new} = \sqrt{{( {x}_{2} - {x}_{A T 1} )}^{2} + {( {y}_{2} - {y}_{A T 1} )}^{2}}$$

In which

$${x}_{2} = ( - {y}_{A} / k + {x}_{A} + {x}_{A} / k + k \cdot {y}_{A} ) / ( 1 / {k}^{2} + 1 )$$

$${y}_{2} = ( 1 / {k}^{2} \cdot {y}_{A} - {x}_{A} / k + {y}_{A T 1} + {x}_{A T 1} / k + {x}_{A} / k - {y}_{A} ) / ( 1 / {k}^{2} + 1 )$$

$$k = ( {y}_{A T 2} - {y}_{A T 1} ) / ( {x}_{A T 2} - {x}_{A T 1} )$$

The new coordinates of the Z axis are:

$${𝓏}_{new} = {𝓏}_{SA} - {𝓏}_{AT 1} .$$

Preferably, the calculation steps of converting the set of laser points obtained from the second scanner to the rock wall scanning surface relative coordinate system in S2 is the same as calculation steps of converting the set of laser points obtained from the first scanner to the rock wall scanning surface relative coordinate system.

Preferably, the specific steps of S3 include the following.

Data obtained from the first scanner and the second scanner are fused in the local coordinate system.

An initial geometric model of the rock surface is formed.

The initial rock wall volume is calculated.

A base volume V0 of the initial rock wall is calculated by integral calculation, in which XZ is used as a base plane, and the Y-axis is used as a height.

Preferably, the specific steps of coarse spraying the shotcrete and performing the three-dimensional modeling calculation of the coarse spray rebound rate in S4 include the following. A three-dimensional modeling calculation of the coarse spray rebound rate is performed

after coarse spraying the shotcrete.

Data obtained from the first scanner and the second scanner are fused in the local coordinate system to form a coarse sprayed shotcrete surface model.

A total volume V1 after coarse spraying the shotcrete is calculated by integral calculation, and an attached square volume V_{coarse spray} is calculated as follows.

$${V}_{coarses spray} = V 1 - V 0$$

A total amount Vs_{coarse spray} of shotcrete sprayed onto a tunnel face is recorded by using a flow meter before and after spraying the shotcrete.

Preferably, the specific steps of fine spraying the shotcrete and performing the three-dimensional modeling calculation of the fine spray rebound rate in S4 include the following.

A three-dimensional modeling calculation of the fine spray rebound rate is performed after fine spraying the shotcrete.

The data obtained from the first scanner and the second scanner are fused in the local coordinate system to form a fine sprayed shotcrete surface model.

A total volume V2 after fine spraying the shotcrete is calculated by integral calculation, and an attached square volume V_{fine spray} is calculated as follows.

$${V}_{fine spray} = V 2 - V 1$$

A total amount Vs_{fine spray} of shotcrete sprayed onto the tunnel face is recorded by using the flow meter before and after spraying the shotcrete.

Preferably, the specific steps of S5 include the following.

The coarse spray rebound rate of the shotcrete is calculated through the formula R_{coarse}=(Vs_{coarse spray}−V_{coarse spray})/Vs_{coarse spray}.

The fine spray rebound rate of the shotcrete is calculated through the formula R_{fine}=(Vs_{fine spray}−V_{fine spray})/Vs_{fine spray}.

It may be seen from the above technical solutions that the disclosure has the following beneficial effects.

In the dynamic monitoring method for the rebound rate of the wet spraying shotcrete according to the disclosure, through scanning the rock surface before wet spraying the shotcrete, the set of laser points obtained from scanning are converted to the rock wall scanning surface relative coordinate system, the data obtained from scanning are fused in the local coordinate system to form the initial geometric model of the rock face, the initial rock wall volume is calculated, the shotcrete is coarse sprayed and fine sprayed, the three-dimensional modeling calculations are performed, and then the coarse spray rebound rate and the fine spray rebound rate are calculated, thereby solving the problem that at present, the monitoring of the rebound rate of the wet spraying shotcrete is mainly roughly evaluated based on the design volume and final usage, based on the known size data of the tunnel design, total shotcrete consumption may be estimated based on the thickness of the shotcrete that needs to be formed after excavation, and when the estimated idealized data does not match the actual usage data, not only it is insufficient in quantification, but also the dynamic rebound rate of the entire wet spraying process is not understood, in which the rebound rate is inaccurately estimated, making it difficult to guide the adjustment of the shotcrete formula and the adjustment of the spray posture and spray speed, and thus the goal of process optimization cannot be achieved.

### BRIEF DESCRIPTION OF THE DRAWINGS

FIG. 1 is a schematic flow chart of a dynamic monitoring method for a rebound rate of wet spraying shotcrete according to the disclosure.

FIG. 2 is a schematic structural diagram of the spatial layout of a first scanner and a second scanner scanning the wet spraying process of the shotcrete according to the dynamic monitoring method for the rebound rate of the wet spraying shotcrete according to the disclosure.

FIG. 3 is a top view of the rock wall coordinate system of the dynamic monitoring method for the rebound rate of the wet spraying shotcrete according to the disclosure.

FIG. 4 is a side view of the rock wall coordinate system of the dynamic monitoring method for the rebound rate of the wet spraying shotcrete according to the disclosure.

FIG. 5 is a schematic diagram of converting of a scanner coordinate system to the rock wall coordinate system according to the dynamic monitoring method for the rebound rate of the wet spraying shotcrete according to the disclosure.

### DESCRIPTION OF THE EMBODIMENTS

The technical solutions in the embodiments of the disclosure will be clearly and completely described below with reference to the accompanying drawings in the embodiments of the disclosure. Certainly, the described embodiments are merely some of the embodiments of the disclosure, rather than all the embodiments. Based on the embodiments of the disclosure, all other embodiments obtained by persons of ordinary skill in the art without creative efforts fall within the scope of protection of the disclosure.

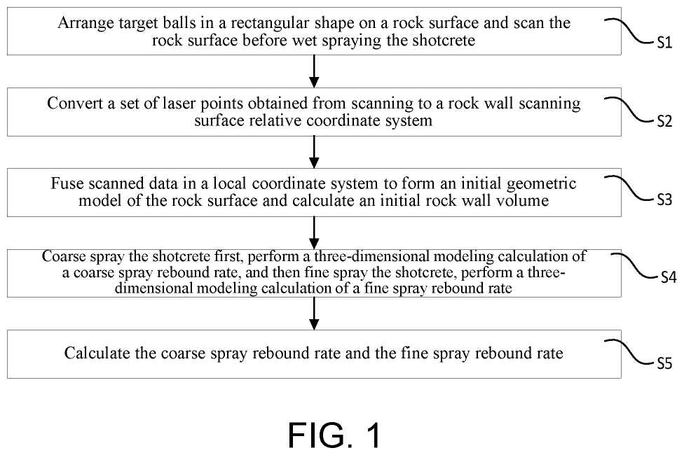

As shown in FIG. 1, a dynamic monitoring method for a rebound rate of a wet spraying shotcrete includes steps as follows.

Step S1. Target balls are arranged in a rectangular shape on the rock surface, and the rock surface is scanned before wet spraying the shotcrete.

Step S2. A set of laser points obtained from scanning are converted to a rock wall scanning surface relative coordinate system.

Step S3. Scanned data are fused in the local coordinate system to form an initial geometric model of the rock surface, and the initial rock wall volume is calculated.

Step S4. First, the shotcrete is coarse sprayed, and a three-dimensional modeling calculation of a coarse spray rebound rate is performed. Then, the shotcrete is fine sprayed, and a three-dimensional modeling calculation of a fine spray rebound rate is performed.

Step S5. Finally, the coarse spray rebound rate and the fine spray rebound rate are calculated.

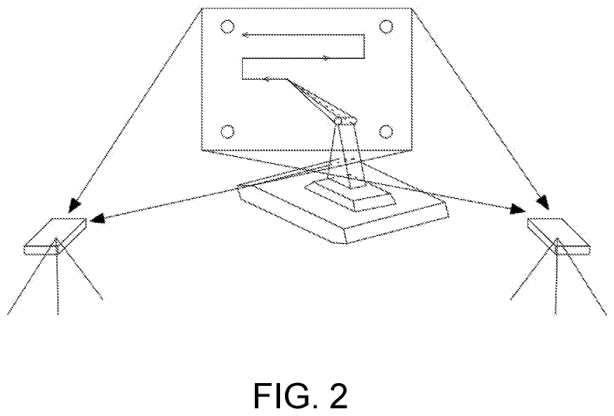

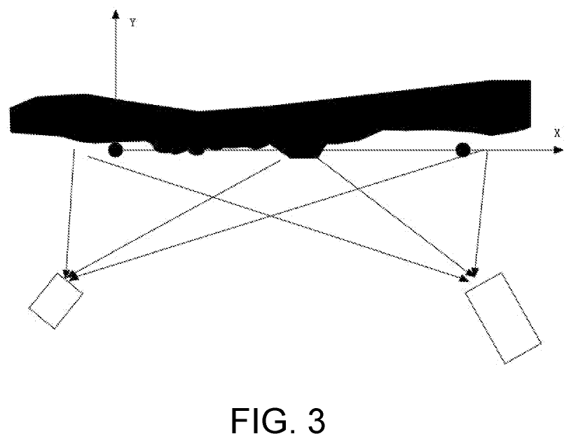

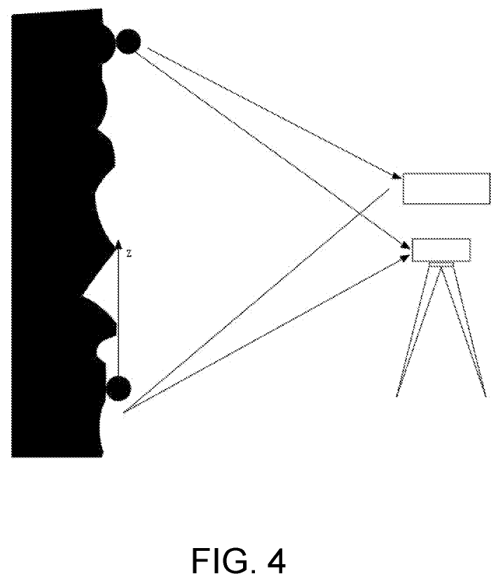

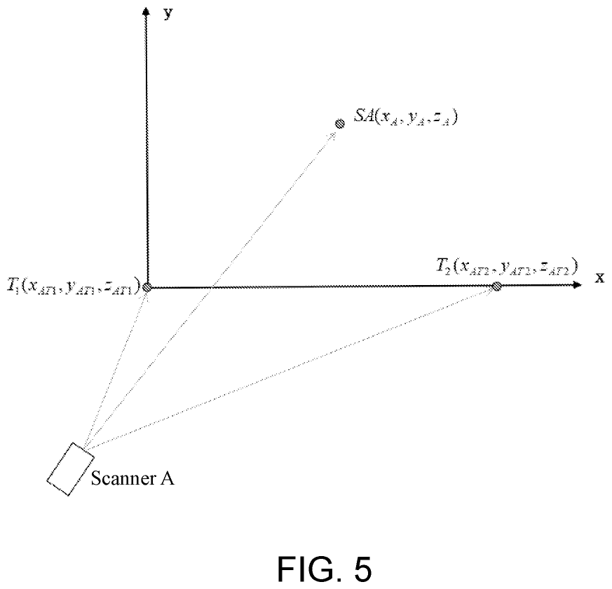

As shown in FIG. 2, FIG. 3, FIG. 4, and FIG. 5, the specific implementation steps are as follows.

First, target balls T1, T2, T3, and T4 are arranged in a rectangular shape on a rock surface to be studied. T1 and T2 are of the same height, while T3 and T4 are vertically above T1 and T2 respectively.

A fixed laser scanner is arranged on each side of the wet spray machine, namely, a first scanner and a second scanner. The first scanner and the second scanner use a 3D laser scanner with data output as a geometric data acquisition device and point to the same area to be scanned comprising the reflective target balls, that is, the area framed by the target balls T1, T2, T3, and T4. For the target balls T1, T2, T3, and T4 as the positioning device of the scanning boundary, spherical reflective targets for surveying and mapping may be used. The scanner scans the target ball to obtain coordinate values of a center point thereof. In order to ensure that the geometry shape of the shotcrete stack may be fully scanned in most cases, the lidar position calculation overlap rate reaches 100% or more.

Then, the rock surface before wet spraying the shotcrete is scanned for the first time by using the first scanner and the second scanner respectively.

Coordinates of the target ball T1 and the target ball T2 obtained by the first scanner are recorded as T1A (X_{AT1}, Y_{AT1}) and T2A (X_{AT2}, Y_{AT2}) respectively. The distance between T1 and T2 are calculated and recorded as L_{T1T2}.

The distance:

$${L}_{T 1 T 2} = \sqrt{{( {X}_{T 1 A} - {X}_{T 2 A} )}^{2} + {( {Y}_{T 1 A} - {Y}_{T 2 A} )}^{2}}$$

The rock wall scanning surface relative coordinate system is established by using T1 and T2. T1 is used as an origin, a direction of T1 pointing to T2 is a positive direction of an X-axis, a vertical direction on a horizontal plane is a positive direction of a Y-axis, a vertical direction upward is a positive direction of a Z-axis, coordinates of T1 are (0,0,0), coordinates of T2 are (L_{T1T2},0,0).

Coordinates of T3 are (0,0,H), coordinates of T4 are (L_{T1T2},0,H), and H is a numerical value where T3 and T4 are higher than T1 and T2.

Then, the set of laser points obtained from the first scanner is converted to the rock wall scanning surface relative coordinate system.

Point coordinates of a measuring point S on the rock wall and shotcrete surface measured by a scanner A are recorded as SA(x_{A}, y_{A}, z_{A}).

Coordinates of each laser measuring point may be converted into the established rock wall coordinate system through the following formula. The calculation formula is as follows.

Coordinates of T1 and T2 are recorded as T₁(x_{AT1}, y_{AT1}, z_{AT1}) and T₂(x_{AT2}, y_{AT2}, z_{AT2}).

Coordinates of a laser scanning point SA in the scanning surface relative coordinate system established in step S2 is SA(x_{new}, y_{new}, z_{new}).

New coordinates of the X-axis are:

$${x}_{new} = \sqrt{( {( {x}_{1} - {x}_{AT 1} )}^{2} + {( {y}_{1} - {y}_{AT 1} )}^{2} )} ,$$

in which

$${x}_{1} = ( k \cdot {y}_{A} + {x}_{A} - k \cdot {x}_{A} - {y}_{A} / k ) / ( {k}^{2} + 1 ) . \\ {y}_{1} = ( {k}^{2} \cdot {y}_{A} + k \cdot {x}_{A} + {y}_{AT 1} - k \cdot {x}_{AT 1} - k \cdot {x}_{A} - {y}_{A} ) / ( {k}^{2} + 1 ) . \\ k = ( {y}_{AT 2} - {y}_{AT 1} ) / ( {x}_{AT 2} - {x}_{AT 1} ) .$$

New coordinates of the Y-axis are:

$${y}_{new} = \sqrt{{( {x}_{2} - {x}_{AT 2} )}^{2} + {( {y}_{2} - {y}_{AT 1} )}^{2}} ,$$

in which

$${x}_{2} = ( - {y}_{A} / k + {x}_{A} + {x}_{A} / k + k \cdot {y}_{A} ) / ( 1 / {k}^{2} + 1 ) . \\ {y}_{2} = ( 1 / {k}^{2} \cdot {y}_{A} - {x}_{A} / k + {y}_{AT 1} + {x}_{AT 1} / k + {x}_{A} / k - {y}_{A} ) / ( 1 / {k}^{2} + 1 ) . \\ k = ( {y}_{AT 2} - {y}_{AT 1} ) / ( {x}_{AT 2} - {x}_{AT 1} ) .$$

New coordinates of the Z axis are:

$${z}_{new} = {z}_{SA} - {z}_{AT 1} .$$

Generally, coordinates obtained by the laser scanner are polar coordinates, SA(θ,φ,D), which are two relative angles and distances. If necessary, the formula of converting SA(θ,φ,D) to SA(x_{A}, y_{A}, z_{A}) may also be attached.

The set of laser points obtained from the second scanner is converted to the rock wall scanning surface relative coordinate system.

Coordinates of each measuring point measured by the second scanner are converted to the established rock wall coordinate system through the formulas by using the same calculation method as the above steps.

Then, the results from the first scanner and the second scanner are fused in the local coordinate system.

An initial geometric model of the rock surface is formed.

The initial rock wall volume is calculated.

A base volume V0 of the initial rock wall is calculated by integral calculation, in which XZ is used as a base plane, and the Y-axis is used as a height.

A three-dimensional modeling calculation of the coarse spray rebound rate is performed after spraying (coarse spraying) the shotcrete for the first time.

The results obtained from the first scanner and the second scanner are fused in the local coordinate system to form a coarse sprayed shotcrete surface model.

A total volume V1 after coarse spraying the shotcrete is calculated by integral calculation, and an attached square volume V_{coarse spray} is calculated as follows.

$${V}_{coarse spray} = V 1 - V 0.$$

A total amount Vs_{coarse spray} of shotcrete sprayed onto a tunnel face is recorded by using a flow meter before and after spraying the shotcrete.

A three-dimensional modeling calculation of the fine spray rebound rate is performed after spraying (fine spraying) the shotcrete for the second time.

The results obtained from the first scanner and the second scanner are fused in the local coordinate system to form a fine sprayed shotcrete surface model.

A total volume V2 after fine spraying the shotcrete is calculated by integral calculation, and an attached square volume V_{fine spray} is calculated as follows.

$${V}_{fine spray} = V 2 - V 1.$$

A total amount Vs_{fine spray} of shotcrete sprayed onto the tunnel face is recorded by using the flow meter before and after spraying the shotcrete.

Finally, the coarse spray rebound rate of the shotcrete is calculated through the formula R_{coarse}=(Vs_{coarse spray}−V_{coarse spray})/Vs_{coarse spray}.

The fine spray rebound rate of the shotcrete is calculated through the formula R_{fine}=(Vs_{fine spray}−V_{fine spray})/Vs_{fine spray}.

Although embodiments of the disclosure have been shown and described, for persons of ordinary skill in the art, it should be noted that various changes, modifications, substitutions, and variations may be made to the embodiments without departing from the principles and spirit of the disclosure. The scope of the disclosure is defined by the appended claims and the equivalents thereof.

What is claimed is:

1. A dynamic monitoring method for a rebound rate of a wet spraying shotcrete, comprising steps as follows:
S1: arranging target balls in a rectangular shape on a rock surface and scanning the rock surface before wet spraying the shotcrete;
S2: converting a set of laser points obtained from scanning to a rock wall scanning surface relative coordinate system;
S3: fusing scanned data in a local coordinate system to form an initial geometric model of the rock surface and calculating an initial rock wall volume;
S4: coarse spraying the shotcrete first, performing a three-dimensional modeling calculation of a coarse spray rebound rate, and then fine spraying the shotcrete, performing a three-dimensional modeling calculation of a fine spray rebound rate; and
S5: calculating the coarse spray rebound rate and the fine spray rebound rate.

2. The dynamic monitoring method for the rebound rate of the wet spraying shotcrete as claimed in claim 1, wherein arranging the target balls in the rectangular shape on the rock surface in S1 comprises:
arranging the target balls T1, T2, T3, and T4 in the rectangular shape on the rock surface to be studied, wherein T1 and T2 are of same height, and T3 and T4 are vertically above T1 and T2 respectively;
arranging a fixed laser scanner each on two sides of a wet spray machine to point at same area to be scanned comprising the reflective target balls.

3. The dynamic monitoring method for the rebound rate of the wet spraying shotcrete as claimed in claim 2, wherein scanning the rock surface before wet spraying the shotcrete in S1 comprises:
scanning the rock surface before wet spraying the shotcrete by using a first scanner and a second scanner respectively;
recording coordinates of the target ball T1 and the target ball T2 obtained by the first scanner as T1A (X_{AT1}, Y_{AT1}) and T2A (X_{AT2}, Y_{AT2}) respectively, calculating a distance between T1 and T2, and recording the distance as L_{T1T2}, wherein
the distance
$${L}_{T 1 T 2} = \sqrt{{( {X}_{T 1 A} - {X}_{T 2 A} )}^{2} + {( {Y}_{T 1 A} - {Y}_{T 2 A} )}^{2}}$$
establishing the rock wall scanning surface relative coordinate system by using T1 and T2, wherein T1 is used as an origin, a direction of T1 pointing to T2 is a positive direction of an X-axis, a vertical direction on a horizontal plane is a positive direction of a Y-axis, a vertical direction upward is a positive direction of a Z-axis, coordinates of T1 are (0,0,0), coordinates of T2 are (L_{T1T2},0,0), coordinates of T3 are (0,0,H), coordinates of T4 are (L_{T1T2},0,H), and H is a numerical value where T3 and T4 are higher than T1 and T2.

4. The dynamic monitoring method for the rebound rate of the wet spraying shotcrete as claimed in claim 3, wherein converting the set of laser points obtained from scanning to the rock wall scanning surface relative coordinate system in S2 comprises:
recording point coordinates of a measuring point S on a rock wall and a shotcrete surface measured by the first scanner as SA(x_{A}, y_{A}, z_{A});
recording the coordinates of T1 as T₁(x_{AT1}, y_{AT1}, z_{AT1}) and the coordinates of T2 as T₂(x_{AT2}, y_{AT2}, z_{AT2});
recording coordinates of a laser scanning point SA in the scanning surface relative coordinate system established in S2 as SA(x_{new}, y_{new}, z_{new});
calculating new coordinates of the X-axis as:
$${x}_{new} = \sqrt{( {( {x}_{1} - {x}_{AT 1} )}^{2} + {( {y}_{1} - {y}_{AT 1} )}^{2} )} ,$$
wherein
$${x}_{1} = ( k \cdot {y}_{A} + {x}_{A} - k \cdot {x}_{A} - {y}_{A} / k ) / ( {k}^{2} + 1 ) \\ {y}_{1} = ( {k}^{2} \cdot {y}_{A} + k \cdot {x}_{A} + {y}_{AT 1} - k \cdot {x}_{AT 1} - k \cdot {x}_{A} - {y}_{A} ) / ( {k}^{2} + 1 ) \\ k = ( {y}_{AT 2} - {y}_{AT 1} ) / ( {x}_{AT 2} - {x}_{AT 1} ) ;$$
calculating new coordinates of the Y-axis as:
$${y}_{new} = \sqrt{{( {x}_{2} - {x}_{AT 1} )}^{2} + {( {y}_{2} - {y}_{AT 1} )}^{2}} ,$$
wherein
$${x}_{2} = ( - {y}_{A} / k + {x}_{A} + {x}_{A} / k + k \cdot {y}_{A} ) / ( 1 / {k}^{2} + 1 ) \\ {y}_{2} = ( 1 / {k}^{2} \cdot {y}_{A} - {x}_{A} / k + {y}_{AT 1} + {x}_{AT 1} / k + {x}_{A} / k - {y}_{A} ) / ( 1 / {k}^{2} + 1 ) \\ k = ( {y}_{AT 2} - {y}_{AT 1} ) / ( {x}_{AT 2} - {x}_{AT 1} ) ; and$$
calculating new coordinates of the Z axis as:
$${z}_{new} = {z}_{SA} - {z}_{AT 1} .$$

5. The dynamic monitoring method for the rebound rate of the wet spraying shotcrete as claimed in claim 4, wherein calculation steps of converting the set of laser points obtained from the second scanner to the rock wall scanning surface relative coordinate system in S2 is the same as calculation steps of converting the set of laser points obtained from the first scanner to the rock wall scanning surface relative coordinate system.

6. The dynamic monitoring method for the rebound rate of the wet spraying shotcrete as claimed in claim 5, wherein fusing the scanned data in the local coordinate system to form the initial geometric model of the rock surface and calculating the initial rock wall volume in S3 comprises:
fusing the data obtained from the first scanner and the second scanner in the local coordinate system;
forming the initial geometric model of the rock surface;
calculating the initial rock wall volume;
calculating a base volume V0 of the initial rock wall by integral calculation, wherein XZ is used as a base plane, and the Y-axis is used as a height.

7. The dynamic monitoring method for the rebound rate of the wet spraying shotcrete as claimed in claim 6, wherein coarse spraying the shotcrete and performing the three-dimensional modeling calculation of the coarse spray rebound rate in S4 comprises:
performing the three-dimensional modeling calculation of the coarse spray rebound rate after coarse spraying the shotcrete;
fusing the data obtained from the first scanner and the second scanner in the local coordinate system to form a coarse sprayed shotcrete surface model;
calculating a total volume V1 after coarse spraying the shotcrete by integral calculation and calculating an attached square volume V_{coarse spray}, wherein
$${V}_{coarse spray} = V 1 - V 0 ;$$
and
recording a total amount Vs_{coarse spray} of the shotcrete sprayed onto a tunnel face by using a flow meter before and after spraying the shotcrete.

8. The dynamic monitoring method for the rebound rate of the wet spraying shotcrete as claimed in claim 7, wherein fine spraying the shotcrete and performing the three-dimensional modeling calculation of the fine spray rebound rate in S4 comprises:
performing the three-dimensional modeling calculation of the fine spray rebound rate after fine spraying the shotcrete;
fusing the data obtained from the first scanner and the second scanner in the local coordinate system to form a fine sprayed shotcrete surface model;
calculating a total volume V2 after fine spraying the shotcrete by integral calculation and calculating an attached square volume V_{fine spray}, wherein
$${V}_{fine spray} = V 2 - V 1 ;$$
and
recording a total amount Vs_{fine spray} of the shotcrete sprayed onto the tunnel face by using the flow meter before and after spraying the shotcrete.

9. The dynamic monitoring method for the rebound rate of the wet spraying shotcrete as claimed in claim 8, wherein calculating the coarse spray rebound rate and the fine spray rebound rate in S5 comprises:
calculatin the coarse spray rebound rate of the shotcrete through a formula R_{coarse}=(Vs_{coarse spray}−V_{coarse spray})/Vs_{coarse spray}; and
calculating the fine spray rebound rate of the shotcrete through a formula R_{fine}=(Vs_{fine spray}−V_{fine spray})/Vs_{fine spray}.
